(19) United States
(12) Patent Application Publication
(10) Pub. No.: US 2008/0188508 A1
(43) Pub. Date: Aug. 7, 2008

(54) Methods and Therapies for Potentiating a Therapeutic Action of an Opioid Receptor Agonist and Inhibiting and/or Reversing Tolerance to Opioid Receptor Agonists

(76) Inventors: Khem Jhamandas, Kingston (CA); Tuan Trang, Toronto (CA)

Correspondence Address: Licata & Tyrrell P.C., 66 E. Main Street, Marlton, NJ, 08053, US

(21) Appl. No.: 12/024,178

(22) Filed: Feb. 1, 2008

Related U.S. Application Data
(60) Provisional application No. 60/887,653, filed on Feb. 1, 2007.

(51) Int. Cl. A61K 31/485; A61P 29/00

(52) U.S. Cl. 514/282

(57) ABSTRACT

Combination therapies of an opioid receptor agonist and a cannabinoid receptor antagonist in an amount effective to potentiate a therapeutic activity of the opioid receptor agonist and/or inhibit, delay, reduce and/or reverse tolerance to the opioid receptor agonist are provided. Also provided are methods for use of these combination therapies in potentiating a therapeutic activity of an opioid receptor agonist and/or inhibiting, delaying or reducing development of acute and/or chronic tolerance to opioid receptor agonists and treating conditions treatable by opioid receptor agonist therapy in a subject. In addition, a method for reversing opioid receptor agonist tolerance and/or restoring therapeutic effect of an opioid receptor agonist in a subject via administration of a cannabinoid receptor antagonist is provided.

This patent application claims the benefit of priority from U.S. Provisional Application Ser. No. 60/887,653, filed Feb. 1, 2007, teachings of which are hereby incorporated by reference in their entirety.

### BACKGROUND OF THE INVENTION

Opioid drugs are indispensable in the clinical management of moderate to severe pain syndromes. Opioids are also used as cough suppressants, in the reduction and/or prevention of diarrhea, and in the treatment of pulmonary edema.

It is well-accepted that the potent analgesic actions of opioids result from interaction with specific receptors present on neurons in the brain, spinal cord and periphery. It is also recognized that there are multiple forms of these receptors. Cloning experiments have identified the existence of three distinct types of receptors, namely mu, delta and kappa. Each type of receptor is a distinct gene product and a 7 transmembrane G-protein coupled receptor (GPCR) (Kieffer et al., Trends in Pharmacol. Science 1999 20:19-26). These receptors are selectively targeted by endogenous opioid peptides and by highly selective agonistic or antagonistic ligands. In particular, endomorphins target mu receptors; enkephalins target delta receptors; and dynorphins target kappa receptors. Pharmacological evidence also suggests the existence of opioid receptor subtypes designated as mu₁ and mu₂, delta₁ and delta₂, and kappa, kappa₂, kappa₃ and kappa₄ (Pasternak and Standifer, Trends in Pharmacol. Science 1995 16:344-350). The molecular structure and/or origin of these opioid receptor subtypes is unclear although alternate processing of gene products (Rossi et al., FEBS Lett 1995 369:192-196; Pan et al., Mol. Pharmacol. 1999 396-403) and/or receptor oligomerization (Jordan and Devi, Nature 1999 399:697-700; George et al., J. Biol. Chem. 2000 275:26128-26135) have been suggested to provide a basis for additional receptor heterogeneity.

While opioids inhibit pain transmission by acting at different levels of the neuraxis, the dorsal spinal cord is recognized as a major site of their action. At this site, opioids inhibit activity of neurons signaling pain by presynaptic and postsynaptic actions. Presynaptically, opioids inhibit the release of several pain neurotransmitters including L-glutamate, calcitonin gene-related peptide (CGRP) and substance P from terminals of the high threshold primary afferents that are driven by the peripheral nociceptive inputs. This effect is attributable to the blockade of the voltage-gated N-type calcium channel (North et al., Proc. Natl Acad. Sci. USA 1987 84:5487-5491; Werz and McDonald, Neuropeptides 1984 5:253-256) regulating the calcium-dependent release of transmitters from nerve terminals. Postsynaptically, opioids hyperpolarize the projection neurons targeted by primary afferents by opening of potassium channels on these neurons. Activation of all opioid receptor types inhibits adenylyl cyclase activity, via a pertussis toxin (PTX)-sensitive mechanism.

The presynaptic and postsynaptic activity of nociceptive neurons is also modulated by several non-opioid receptors that operationally behave as opioid receptors.

The development of tolerance, at least with respect to opioid receptor agonists, has been attributed to multiple factors (Jhamandas et al., Pain Res. Manag. 2000 5:25-32). Recent studies suggest that tolerance may result from the paradoxical stimulatory actions of opioids that are exerted at very low doses and that may progressively overwhelm the inhibitory effects contributing to analgesia (Crain and Shen, Trends in Pharm. Sci. 1990 11:177-81). The excitatory actions of opioids are blocked by opioid receptor antagonists (e.g. naloxone or naltrexone) when administered at ultra-low doses 50 to 100,000-fold lower than doses of opioid receptor antagonists blocking or inhibiting the classical opioid actions (Crain and Shen, Proc. Natl Acad. Sci USA 1995 92:10540-10544). Such ultra-low doses of the opioid receptor antagonist, naltrexone, paradoxically increase opioid analgesia, inhibit development of chronic opioid tolerance and reverse established tolerance (Powell et al., J. Pharmacol. Exp. Ther. 2002 300:588-596). The hypothesis underlying these actions is that the latent excitatory effects of an opioid produce hyperalgesia which is progressive and eventually overcomes the analgesia produced by classical opioid doses. However, clinical use of opioid receptor antagonists carries the risk of potential loss of the analgesic response.

Chronic morphine treatment has been shown to increase both the expression and release of CGRP in the spinal cord (Powell et al. Br. J. Pharmacol. 2000 131:875-884; Gardell et al. J. Neurosci. 2002 22:6747-6755). The resulting action of the neuropeptide on its receptors contributes to the development and maintenance of opioid analgesic tolerance, as well as the manifestations of the opioid withdrawal syndrome.

Traditionally, cannabinoid-1 (CB1)-receptors have been viewed as inhibitory to the cAMP pathway. However, studies in Chinese hamster ovary cells indicate a dual coupling of these receptors to the cAMP signaling pathway via Gᵢ-inhibitory and Gₛ-excitatory proteins (Calandra et al., 1999).

Cross-tolerance between opioid and cannabinoid agonist-induced analgesia has been shown (Thorat, S. N. and Bhargava, H. N. Eur. J. Pharmacol. 1994 260:5-13; Pontieri et al. Eur. J. Pharmacol. 2001 421:R1-R3).

### SUMMARY OF THE INVENTION

An aspect of the present invention is a composition comprising an opioid receptor agonist, in an amount effective to produce a therapeutic effect, and a cannabinoid-receptor antagonist, in an amount effective to potentiate the therapeutic effect of the opioid receptor agonist and/or inhibit, delay and/or reverse tolerance to the opioid receptor agonist. Compositions of the present invention provide useful therapeutic agents for management of pain including, but not limited to, acute and/or chronic post-surgical pain, obstetrical pain, acute and/or chronic inflammatory pain, pain associated with conditions such as multiple sclerosis and/or cancer, pain associated with trauma, pain associated with migraines, neuropathic pain, central pain and chronic pain syndromes of a non-malignant origin such as chronic back pain. Compositions of the present invention are also useful as cough suppressants, in reduction and/or prevention of diarrhea, in treatment of pulmonary edema and in alleviating physical dependence and/or addiction to opioid receptor agonists.

Another aspect of the present invention is a method for potentiating a therapeutic activity of an opioid receptor agonist which comprises administering to a subject in combination with an opioid receptor agonist a cannabinoid receptor antagonist in an amount effective to potentiate a therapeutic activity of the opioid receptor agonist and/or inhibit, delay and/or reverse tolerance to the opioid receptor agonist.

Another aspect of the present invention is a method for potentiating a biological action of an endogenous opioid receptor agonist in a subject which comprises administering to the subject a cannabinoid receptor antagonist in an amount effective to potentiate the biological action of the endogenous opioid receptor agonist.

Another aspect of the present invention is a method for inhibiting development of acute tolerance to a therapeutic action of an opioid receptor agonist in a subject which comprises administering to a subject in combination with an opioid receptor agonist a cannabinoid receptor antagonist in an amount effective to potentiate a therapeutic activity of the opioid receptor agonist and/or inhibit, delay and/or reverse tolerance to the opioid receptor agonist.

Another aspect of the present invention is a method for inhibiting development of chronic tolerance to a therapeutic action of an opioid receptor agonist in a subject which comprises administering to a subject in combination with an opioid receptor agonist a cannabinoid receptor antagonist in an amount effective to potentiate a therapeutic activity of the opioid receptor agonist and/or inhibit, delay and/or reverse tolerance to the opioid receptor agonist.

Another aspect of the present invention is a method for reversing tolerance to a therapeutic action of an opioid receptor agonist and/or restoring therapeutic potency of an opioid receptor agonist in a subject tolerant to a therapeutic action of an opioid receptor agonist which comprises administering a cannabinoid receptor antagonist to a subject receiving an opioid receptor agonist in an amount effective to potentiate a therapeutic activity of the opioid receptor agonist and/or inhibit, delay and/or reverse tolerance to the opioid receptor agonist.

Another aspect of the present invention is a method for treating a subject suffering from a condition treatable with an opioid receptor agonist comprising administering to the subject an opioid receptor agonist in an amount effective to produce a therapeutic effect and a cannabinoid receptor antagonist in an amount effective to potentiate a therapeutic activity of the opioid receptor agonist and/or inhibit, delay and/or reverse tolerance to the opioid receptor agonist.

The above methods are useful for treating subjects suffering from conditions including, but not limited to, pain, coughs, diarrhea, pulmonary edema, addiction and physical dependence to opioid receptor agonists. It is understood that such treatment may also be commenced prior to such suffering (i.e., prophylactically, when the subject is at risk for such suffering).

Yet a further aspect of the present invention in each of the above methods is that the cannabinoid receptor antagonist is administered or formulated in an amount which potentiates a therapeutic activity of the opioid receptor agonist and/or inhibits, delays and/or reverses tolerance to the opioid receptor agonist, and that the amount of the cannabinoid receptor antagonist, alone or in combination with the opioid receptor agonist, does not elicit a substantial undesirable side effect.

Although this invention is described in detail with reference to preferred embodiments thereof, these embodiments are offered to illustrate but not to limit the invention. It is possible to make other embodiments that employ the principles of the invention and that fall within its spirit and scope as defined by the claims appended hereto.

### BRIEF DESCRIPTION OF THE FIGURES

FIGS. 1A and 1B are line graphs depicting the time-course of the antinociceptive effect of acute intrathecal morphine alone and in combination with the CB1 receptor antagonist, AM-251, on the development of acute morphine tolerance in a rat tail-flick (FIG. 1A) and rat paw-pressure (FIG. 1B) tests. Intrathecal drug injection was given at 0, 90 and 180 minutes. Control groups received either saline or single injection of morphine at 0 minutes followed by saline injections at 90 and 180 minutes. Nociceptive testing was performed at 30 minute intervals over 4 hours. The data is presented as mean±s.e. mean, n=4-8 animals per treatment group. Significant differences from the action of morphine alone are indicated by *(P<0.05) and **(P<0.01).

FIGS. 2A and 2B are line graphs depicting dose-response curves of the analgesic effects of intrathecal morphine following acute morphine treatment in the rat tail-flick (FIG. 2A) and rat paw-pressure (FIG. 2B) tests. On day 2, animals were administered ascending doses of morphine every 30 minutes until a maximal level of antinociception was achieved in both nociceptive tests. The data is expressed as mean±s.e. mean, n=4-8 animals per treatment group.

FIGS. 3A and 3B are line graphs depicting the time-course of the antinociceptive effect of daily intrathecal morphine alone and in combination with AM-251 in the rat tail-flick (FIG. 3A) and paw-pressure (FIG. 3B) tests. Nociceptive testing was performed 30 minutes following each injection. The data are expressed as mean±s.e. mean, n=4-8 animals per treatment group. Significant differences from morphine-only treated animals are indicated by *(p<0.05) and **(p<0.01).

FIGS. 4A and 4B are line graphs depicting dose-response curves of the analgesic effects of acute intrathecal morphine following 5-day chronic treatment in rat tail-flick (FIG. 4A) and paw-pressure (FIG. 4B) tests. On day 6, animals were administered ascending doses of morphine every 30 minutes until a maximal level of antinociception was achieved in both nociceptive tests. The data are expressed as mean±s.e. mean, n=4-8 animals per treatment group.

FIGS. 5A and 5B are line graphs depicting the time-course of the effects of intrathecal AM-251 on established morphine tolerance in rat tail-flick (FIG. 5A) and rat paw-pressure (FIG. 5B) tests. Tolerance to the antinociceptive action of spinal morphine was established by daily injection on days 1-5. Daily intervention with intrathecal AM-251 occurred on days 6-10. Nociceptive testing was performed 30 minutes following each injection. The data are expressed as mean±s.e. mean, n=5 animals per treatment group. Significant differences from morphine-only treated animals are indicated by *(p<0.05) and **(p<0.01).

FIGS. 6A and 6B are line graphs depicting dose-response curves of the analgesic effects of acute intrathecal morphine following 10 day chronic treatment in rat tail-flick (FIG. 6A) and paw-pressure (FIG. 6B) tests. On day 11, animals were administered ascending doses of morphine every 30 minutes until a maximal level of antinociception was achieved in both nociceptive tests. The data are expressed as mean±s.e. mean, n=5 animals per treatment group.

FIGS. 7A through 7F are photomicrographs of CGRP-immunoreactive neurons in the spinal lumbar dorsal horn of rats following intrathecal injection of saline (FIG. 7A), morphine (15 μg) 4 hours (FIG. 7B), morphine (15 μg) 5 days (FIG. 7C), morphine and AM-251 (3.2 μg) 5 days (FIG. 7D), morphine (15 μg) 10 days (FIG. 7E), and morphine 5 days then morphine and AM-251 (3.2 μg) for another 5 days (FIG. 7F). The scale bar for these photomicrographs is 100 μm.

FIG. 8A and 8B depict the number of CGRP-immunoreactive neurons in adult rat dorsal root ganglion cultures after 5 day treatment with morphine alone or in combination with AM-251. The bar graph of FIG. 8A represents the mean number of CGRP-immunoreactive neurons counted in each group expressed as a percent of the CGRP-immunoreactive neurons observed in the control (drug-free) group. Corresponding photomicrographs for the control (Drug free) group and groups administered morphine (20 μM), AM-251 (10 μM), morphine and AM-251 (1 μM), morphine and AM-251 (5 μM) and morphine and AM-251 (10 μM) are depicted in FIG. 8B. Significant differences from the control group are depicted by *(p<0.05) and **(P<0.01). n=4 for each treatment group.

### DETAILED DESCRIPTION OF THE INVENTION

Mobilization of spinal CGRP contributing to the behavioral and neurochemical responses elicited during naloxone-precipitated morphine withdrawal has been shown to be partially mediated by CB₁-receptors (Trang et al. Pain 2006 126(1-3):256-71 2006). Recent evidence also suggests that CB₁-receptors form heteromeric receptor complexes with mu-opioid receptors and co-activation of this receptor-complex leads to reciprocal inhibition of receptor signaling (Rios et al. Br. J. Pharmacol. 2006 148:387-395).

It has now been found that administration of a cannabinoid receptor antagonist potentiates opioid receptor agonist analgesia and inhibits, delays, reduces and/or reverses the development of acute or chronic tolerance to opioid receptor agonists. The present invention provides new combination therapies for potentiating a therapeutic activity of an opioid receptor agonist and/or inhibiting, delaying or reducing development of and/or reversing, at least partially, chronic and/or acute tolerance to an opioid receptor agonist involving co-administration of an opioid receptor agonist with a cannabinoid receptor antagonist. An aspect of the present invention thus relates to compositions comprising an opioid receptor agonist and a cannabinoid receptor antagonist. Another aspect of the present invention relates to methods for potentiating a therapeutic activity of an opioid receptor agonist and/or effectively inhibiting, delaying or reversing the development of acute as well as chronic tolerance to a therapeutic action of an opioid receptor agonist by co-administering the opioid receptor agonist with a cannabinoid receptor antagonist. The new combination therapies of the present invention are expected to be useful in optimizing the use of opioid drugs in various applications including but not limited to: pain management, e.g., management of acute or chronic post-surgical pain, obstetrical pain, acute or chronic inflammatory pain, pain associated with conditions such as multiple sclerosis or cancer, pain associated with trauma, pain associated with migraines, neuropathic pain, and central pain; management of chronic pain syndrome of a non-malignant origin such as chronic back pain; cough suppression; reducing and/or preventing diarrhea; treating pulmonary edema; and alleviating addiction or physical dependence to opioid receptor agonists. In a preferred embodiment, the combination therapies of the present invention are used in pain management.

As used herein, the term “co-administer” encompasses administering two or more agents. The two or more agents may be administered at the same time (i.e., simultaneously), or at different times. Simultaneous administration may include administering the two or more agents separately, as separate dosage units, or combined in a single dosage unit. When the two or more agents are co-administered at different times, one agent may be administered before or after the other agent(s). For example, the cannabinoid receptor antagonist may be administered prior to, simultaneously with, or after administration of the opioid receptor agonist.

Cannabinoid receptor antagonists useful in the combination therapies and methods of the present invention include any compound that partially or completely reduces, inhibits, blocks, inactivates and/or antagonizes the binding of a cannabinoid receptor agonist to its receptor to any degree and/or the activation of a cannabinoid receptor to any degree. Thus, the term cannabinoid receptor antagonist is also meant to include compounds that antagonize the agonist in a competitive, irreversible, pseudo-irreversible and/or allosteric mechanism.

Cannabinoid receptor antagonists useful in the combination therapies and methods of the present invention include, but are in no way limited to, antagonists of cannabinoid 1 (CB1) receptors, antagonists of cannabinoid 2 (CB2) receptors, and antagonists of both CB1 and CB2 receptors. In a preferred embodiment, the cannabinoid receptor antagonist is a CB1 receptor antagonist. Examples of cannabinoid receptor antagonists useful in the present invention include, but are in no way limited to, SR 141716 (Sanofi-Aventis, Paris, France), AM-251 (Tocris Cookson, Bristol, UK), AM281 (Tocris Cookson, Bristol, UK) LY320135 (Eli Lilly, Inc. Indiana), and SR 144528 (Rinaldi-Carmona et al. J Pharmacol Exp Ther 1998 284:644-650). Exemplary cannabinoid receptor antagonists useful in the present invention are also set forth in U.S. Pat. Nos. 6,825,209, 5,547,524, and 6,916,838 and published U.S. Patent Application 2005/0014786. In some embodiments, preferred cannabinoid receptor antagonists are SR 141716 and LY320135. The cannabinoid receptor antagonist is included in the compositions and administered in the methods of the present invention at an amount effective to potentiate a therapeutic effect of an opioid receptor agonist and/or inhibit, delay or reverse tolerance to an opioid receptor agonist.

Compositions of the present invention as well as methods described herein for their use may comprise more than one cannabinoid receptor antagonist alone, or more than one cannabinoid receptor antagonist in combination with one or more opioid receptor agonists. These agents may be co-administered as set forth herein.

The cannabinoid receptor antagonist is included in the compositions and administered in the methods of the present invention at a dose effective at potentiating a therapeutic activity of an opioid receptor agonist and/or inhibiting, delaying or reversing tolerance to the opioid receptor agonist. Exemplary doses used in experiments described herein contain an amount of cannabinoid receptor antagonist sufficient to produce a blockade of the cannabinoid receptor.

As used herein, the term “amount” is intended to refer to the quantity of cannabinoid receptor antagonist and/or opioid receptor agonist administered to a subject. The term “amount” encompasses the term “dose” or “dosage”, which is intended to refer to the quantity of cannabinoid receptor antagonist and/or opioid receptor agonist administered to a subject at one time or in a physically discrete unit, such as, for example, in a pill, injection, or patch (e.g., transdermal patch). The term “amount” also encompasses the quantity of cannabinoid receptor antagonist and/or opioid receptor agonist administered to a subject, expressed as the number of molecules, moles, grams, or volume per unit body mass of the subject, such as, for example, mol/kg, mg/kg, ng/kg, ml/kg, or the like, sometimes referred to as concentration administered.

In accordance with the invention, administration to a subject of a given amount of cannabinoid receptor antagonist and/or opioid receptor agonist results in an effective concentration of the antagonist and/or agonist in the subject's body. As used herein, the term “effective concentration” is intended to refer to the concentration of cannabinoid receptor antagonist and/or opioid receptor agonist in the subject's body (e.g., in the blood, plasma, or serum, at the target tissue(s), or site(s) of action) capable of producing a desired therapeutic effect. The effective concentration of cannabinoid receptor antagonist and/or opioid receptor agonist in the subject's body may vary among subjects and may fluctuate within a subject over time, depending on factors such as, but not limited to, the condition being treated, genetic profile, metabolic rate, biotransformation capacity, frequency of administration, formulation administered, elimination rate, and rate and/or degree of absorption from the route/site of administration.

For at least these reasons, for the purpose of this disclosure, administration of cannabinoid receptor antagonist and/or opioid receptor agonist is conveniently provided as amount or dose of cannabinoid receptor antagonist or opioid receptor agonist. The amounts, dosages, and dose ratios provided herein are exemplary and may be adjusted, using routine procedures such as dose titration, to provide an effective concentration.

In one embodiment the amount of cannabinoid receptor antagonist administered potentiates a therapeutic activity of an opioid receptor agonist and/or inhibits, delays or reverses tolerance to the opioid receptor agonist. Thus, the effective concentration of a cannabinoid receptor antagonist is a concentration in the body which potentiates a therapeutic activity of an opioid receptor agonist and/or inhibits, delays or reverses tolerance to the opioid receptor agonist. Preferably, the amount of cannabinoid receptor antagonist administered potentiates a therapeutic activity of an opioid receptor agonist and/or inhibits, delays or reverses tolerance to the opioid receptor agonist without the amount of the cannabinoid receptor antagonist, alone or in combination with the opioid receptor agonist, eliciting a substantial undesirable side effect.

Effective doses useful in the present invention for cannabinoid receptor antagonists in combination with opioid receptor agonists can be determined routinely by those skilled in the art in accordance with the known effective concentrations for cannabinoid receptor antagonists and opioid receptor agonists and the methodologies described herein and will depend upon the cannabinoid receptor antagonist and the route of administration selected for the combination therapy. Further, those skilled in the will recognize that interspecies pharmacokinetic scaling can be used to study the underlining similarities (and differences) in drug disposition among species, to predict drug disposition in an untested species, to define pharmacokinetic equivalence in various species, and to design dosage regimens for experimental animal models, as discussed in Mordenti, Man versus Beast: Pharmacokinetic Scaling in Mammals, 1028, Journal of Pharmaceutical Sciences, Vol. 75, No. 11, Nov. 1986.

For example, for the cannabinoid receptor antagonist AM-251, intrathecal administration of 1.6 and 3.2 μg was demonstrated to be effective in potentiating the analgesic effect of the opioid receptor agonist and/or inhibiting tolerance to the opioid receptor agonist. Accordingly, it is expected that systemic dosing in the range of about 0.3 to 100 mg/kg of AM-251 will produce similar effects in patients. Further, AM-251 has been shown to have pharmacological activities at systemically administered doses as low as 10 fg/kg to 100 ng/kg (Gholizadeh et al. Neuropharmacology 2007 53(6):763-70). Thus, lower doses of AM-251 in the range of 10 fg/kg up to 0.3 mg/kg may be effective as well.

As another example, the cannabinoid receptor antagonist SR 141716 was recently granted marketing authorization under the tradename ACOMPLIA (rimonabant) at a dose of 20 mg/day orally. Further, doses of SR 141716 in the range of 0.3 to 3 mg/kg i.p. have been demonstrated to exhibit pharmacological activities (Caillé et al. Eur. J. Neurosci. 2003 18:3145-3149). Accordingly, it is expected that systemic doses in the range of 0.03 to 100 mg/kg, preferably 0.2 to 10 mg/kg, of this cannabinoid receptor antagonist will be effective in potentiating the analgesic effect of the opioid receptor agonist and/or inhibiting tolerance to the opioid receptor agonist

Similar effective dose ranges for other cannabinoid receptor antagonists can be determined routinely based upon similar experiments as described herein and/or doses demonstrated to exhibit pharmacological activities in the literature.

By “substantial undesirable side effect” as used herein it is meant a response in a subject to the cannabinoid receptor antagonist other than potentiating the therapeutic action of the opioid receptor agonist and/or inhibiting, delaying or reversing tolerance to the opioid receptor agonist which can not be controlled in the subject and/or endured by the subject and/or could result in discontinued treatment of the subject with the combination therapies and methods of the present invention.

Examples of such side effects include, but are not limited to, tolerance, physical and/or psychological dependence, addiction, sedation, euphoria, dysphoria, memory impairment, hallucination, depression, headache, hyperalgesia, constipation, insomnia, body aches and pains, change in libido, respiratory depression and/or difficulty breathing, nausea and vomiting, pruritus, dizziness, fainting (i.e. syncope), nervousness and/or anxiety, irritability, psychoses, tremors, changes in heart rhythm, decrease in blood pressure, elevated blood pressure, elevated heart rate, risk of heart failure, temporary muscle paralysis and diarrhea.

Opioid receptor agonists useful in the combination therapies and methods of the present invention include any compound (either endogenous or exogenous to the subject) that binds to and/or activates and/or agonizes an opioid receptor to any degree and/or stabilizes the opioid receptor in an active or inactive conformation. Thus, by the term opioid receptor agonist it is meant to include partial agonists, inverse agonists, as well as full agonists of an opioid receptor. By opioid receptor agonist it is also meant to be inclusive of compounds that enhance the activity of opioid receptor agonist compounds produced within the body, as well as exogenous opioid receptor agonists (i.e., synthetic or naturally-occurring). Preferred opioid receptor agonists used in the present invention are partial or full agonists of the mu, delta, and/or kappa opioid receptors. Preferred opioid receptor agonists also include compounds from the opioid class of drugs, and more preferably opioids which act as analgesics. Examples of opioid receptor agonists useful in the present invention include, but are in no way limited to morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2,D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, tramadol, dezocine, etorphine, tilidine, loperamide, diphenoxylate, paregoric and nalorphine.

Compositions of the present invention as well as methods described herein for their use may comprise more than one opioid receptor agonist and/or more than one cannabinoid receptor antagonist, formulated and/or administered in various combinations.

A preferred combination of opioid receptor agonist and cannabinoid receptor antagonist used in the present invention is an opioid and a CB1 receptor antagonist. Examples include, but are not limited to, morphine or a related opioid and AM-251 or SR 141716 or another CB1 receptor antagonist.

The dose of opioid receptor agonist included in the compositions of the present invention and used in the methodologies described herein is an amount that achieves an effective concentration and/or produces a desired therapeutic effect. For example, such a dosage may be an amount of opioid receptor agonist well known to the skilled artisan as having a therapeutic action or effect in a subject. Dosages of opioid receptor agonist producing, for example, an analgesic effect can typically range between about 0.02 mg/kg to 100 mg/kg, depending upon, but not limited to, the opioid receptor agonist selected, the route of administration, the frequency of administration, the formulation administered, and/or the condition being treated. Further, since co-administration of an opioid receptor agonist with a cannabinoid receptor antagonist potentiates the analgesic effect of the opioid receptor agonist, the amount or dose of opioid receptor agonist effective at producing a therapeutic effect may be lower than when the opioid receptor agonist is administered alone.

For purposes of the present invention, by “therapeutic effect” or “therapeutic activity” or “therapeutic action” it is meant a desired pharmacological activity of an opioid receptor agonist useful in the inhibition, reduction, prevention or treatment of a condition routinely treated with an opioid receptor agonist. Examples include, but are not limited to, pain, coughs, diarrhea, pulmonary edema and physical and/or psychological addiction to opioid receptor agonists. By these terms it is meant to include a pharmacological activity measurable as an end result, i.e. alleviation of pain or cough suppression, as well as a pharmacological activity associated with a mechanism of action linked to the end desired result. In a preferred embodiment, the “therapeutic effect” or “therapeutic activity” or “therapeutic action” is alleviation or management of pain.

For purposes of the present invention, by “potentiate”, it is meant that administration of the cannabinoid receptor antagonist enhances, extends or increases, at least partially, the therapeutic activity of an opioid receptor agonist and/or results in a decreased amount of opioid receptor agonist being required to produce a desired therapeutic effect. Thus, as will be understood by the skilled artisan upon reading this disclosure, the amount of opioid receptor agonist included in the combination therapy of the present invention may be decreased as compared to an established amount of the opioid receptor agonist when administered alone. The amount of the decrease for other opioid receptor agonists can be determined routinely by the skilled artisan based upon ratios described herein for morphine and AM-251. By potentiate it is also meant to include any enhancement, extension or increase in therapeutic activity of an agent whose therapeutic activity is dependent on increased synthesis or release of an endogenous opioid receptor agonist.

This decrease in required amount of opioid receptor agonist to achieve the same or similar therapeutic benefit may decrease any unwanted side effects associated with opioid receptor agonist therapy. Thus, the combination therapies of the present invention also provide a means for decreasing unwanted side effects of opioid receptor agonist therapy.

By “tolerance” as used herein, it is meant a loss of level of drug-induced response and drug potency and is produced by many opioid receptor agonists, and particularly opioids. Chronic or acute tolerance can be a limiting factor in the clinical use of opioid drugs as opioid potency is decreased upon exposure to the opioid. By “chronic tolerance” it is meant a decrease in level of drug-induced response and drug potency which can develop after drug exposure over several or more days. “Acute tolerance” is a loss in drug potency which can develop after drug exposure over several hours (Fairbanks and Wilcox J. Pharmacol. Exp. Therapeutics. 1997 282:1408-1417; Kissin et al. Anesthesiology 1991 74:166-171). Loss of opioid drug potency may also be seen in pain conditions such as neuropathic pain without prior opioid drug exposure as neurobiological mechanisms underlying the genesis of tolerance and neuropathic pain are similar (Mao et al. Pain 1995 61:353-364). This is also referred to as acute tolerance. Tolerance has been explained in terms of opioid receptor desensitization or internalization although exposure to morphine, unlike most other mu opioid receptor agonists, does not produce receptor internalization. It has also been explained on the basis of an adaptive increase in levels of pain transmitters such as glutamic substance P or CGRP. Inhibition of tolerance and maintenance of opioid potency are important therapeutic goals in pain management which, as demonstrated herein, are achieved via the combination therapies of the present invention.

One skilled in the art would know which combination therapies would work to potentiate a therapeutic activity of an opioid receptor agonist and/or inhibit, delay or reverse acute or chronic opioid receptor agonist tolerance upon co-administration of a cannabinoid receptor antagonist based upon the disclosure provided herein. For example, any given combination of opioid receptor agonist and cannabinoid receptor antagonist may be tested in animals using one or more available tests, including, but not limited to, tests for analgesia such as thermal, mechanical and the like, or any other tests useful for assessing antinociception as well as other therapeutic actions of opioid receptor agonists. Non-limiting examples for testing analgesia include the thermal rat tail flick and mechanical rat paw pressure antinociception assays.

The ability of exemplary combination therapies of the present invention to potentiate a therapeutic activity of an opioid receptor agonist and inhibit, delay or reverse acute or chronic opioid receptor agonist tolerance upon co-administration of a cannabinoid receptor antagonist was demonstrated in tests of both thermal (rat tail flick) and mechanical (rat paw pressure) antinociception. In these experiments, the opioid receptor agonist was the opioid morphine. The cannabinoid receptor antagonist was AM-251, a selective CB1-receptor antagonist/inverse agonist. The dose of AM-251 used in these experiments was previously shown to selectively block the anandamide-induced antinociception but have no influence on acute morphine-induced analgesia (Trang et al. Pain 2006 126(1-3):256-71). Additionally, in the experiments intrathecal injection of AM-251 alone did not change nociceptive responses and had no effect on behavioral or biochemical indices of opioid tolerance.

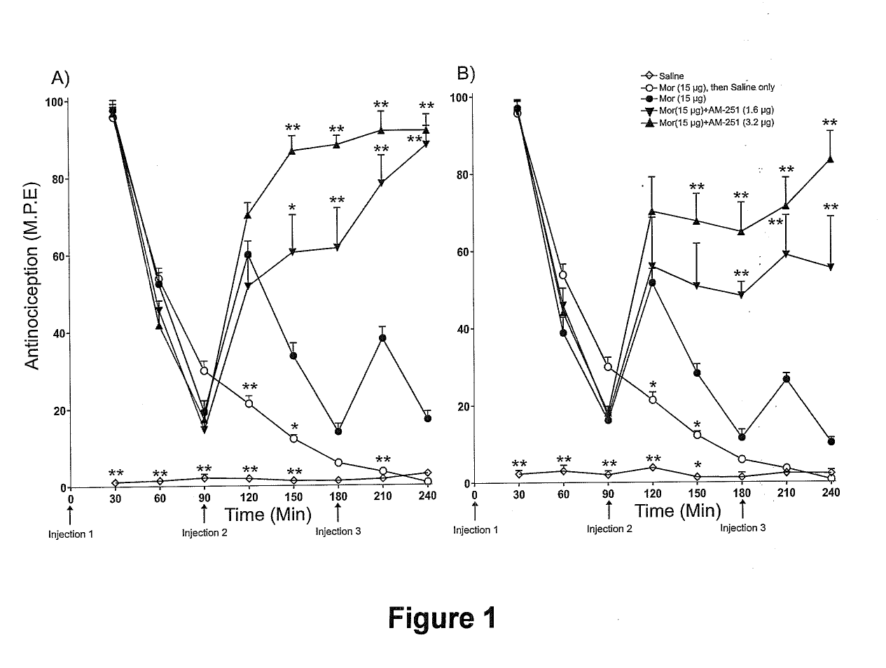

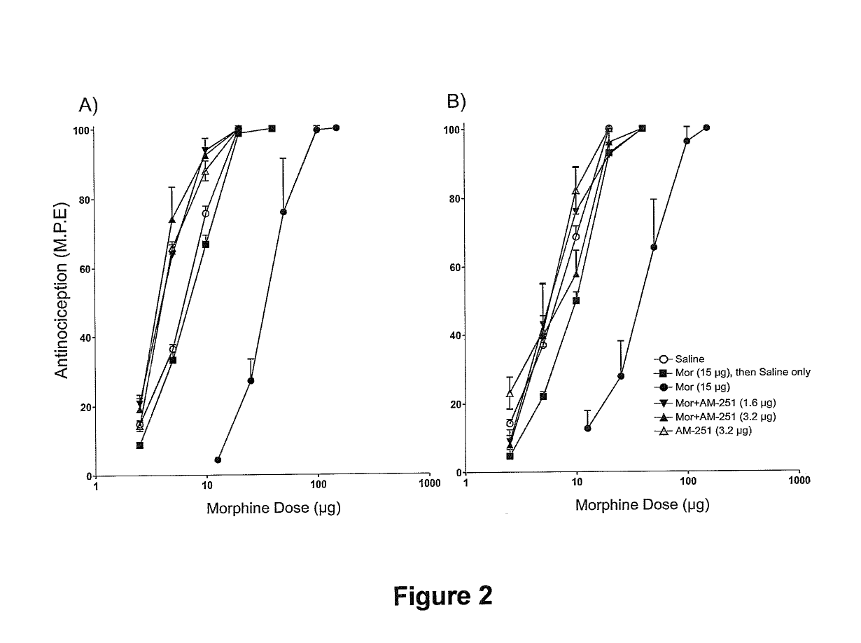

The behavioral effects of AM-251 on acute morphine tolerance were examined in rats. Results from these experiments are depicted in FIGS. 1 and 2. As shown therein, intrathecal injection of morphine (15 μg; n=8) produced a maximal antinociceptive response at 30 minutes post-injection and returned to baseline by 90 minutes in both the tail-flick (FIG. 1A) and paw-pressure test (FIG. 1B). Subsequent intrathecal injections of morphine (15 μg) elicited a diminished antinociceptive response. Specifically, relative to the first injection of morphine, antinociception elicited by the second injection was reduced by 38% and 45% in the tail-flick (FIG. 1A) and paw-pressure (FIG. 1B) tests, respectively. Likewise, antinociception produced by the third injection of morphine was 60% and 45% lower in the tail-flick (FIG. 1A) and paw-pressure (FIG. 1B) tests, respectively, compared to the first intrathecal morphine injection in the morphine-naïve animals. The decline in maximal antinociceptive effect reflects a rapid development of tolerance to the acute actions of morphine. In the positive control group, a single intrathecal injection of morphine (15 μg; n=4) was given at the onset of the experiment followed by an injection of saline at 90 and 180 minutes. As expected, injection of morphine produced maximal antinociception whereas subsequent injections of saline elicited responses comparable to saline-only treated control group, indicating that baseline response in the tail-flick (FIG. 1A) and paw-pressure (FIG. 1B) tests was not altered during the time-course of the experiments. Co-administration of the cannabinoid receptor antagonist AM-251 (1.6 μg; n=6) with intrathecal morphine attenuated the loss in morphine antinociception. At a higher dose, intrathecal injection of AM-251 (3.2 μg; n=7) resulted in a pronounced inhibition of acute morphine tolerance. Intrathecal injections of AM-251 alone, however, had no effect on baseline antinociceptive responses in either the tail-flick or paw-pressure test.

On day 2, animals were given ascending doses of morphine at 30-minute periods until a maximal level of antinociception was reached in both the tail-flick (FIG. 2A) and paw-pressure tests (FIG. 2B). ED₅₀ values for morphine were derived from the constructed dose-response curves. These values are shown in Table 1.

Further, as illustrated in FIG. 2A and 2B, repeated intrathecal injections of morphine (15 μg) produced a rightward shift in the cumulative dose-response curve reflecting a substantial loss of agonist potency. This was also indicated by a 5.9-fold (P<0.01) and 5.6-fold (P<0.01) increase in ED₅₀ value in the tail-flick and paw-pressure tests, respectively, as compared to saline-only controls. In the positive control group receiving a single injection of morphine followed by subsequent saline injections, ED₅₀ values were not statistically different from saline-only control. Co-treatment of AM-251 with morphine suppressed the increase in ED₅₀ value and prevented the rightward shift in the dose-response curve. Administration of AM-251 (3.2 μg) alone did not change morphine ED₅₀ value from that obtained from saline-only treated controls.

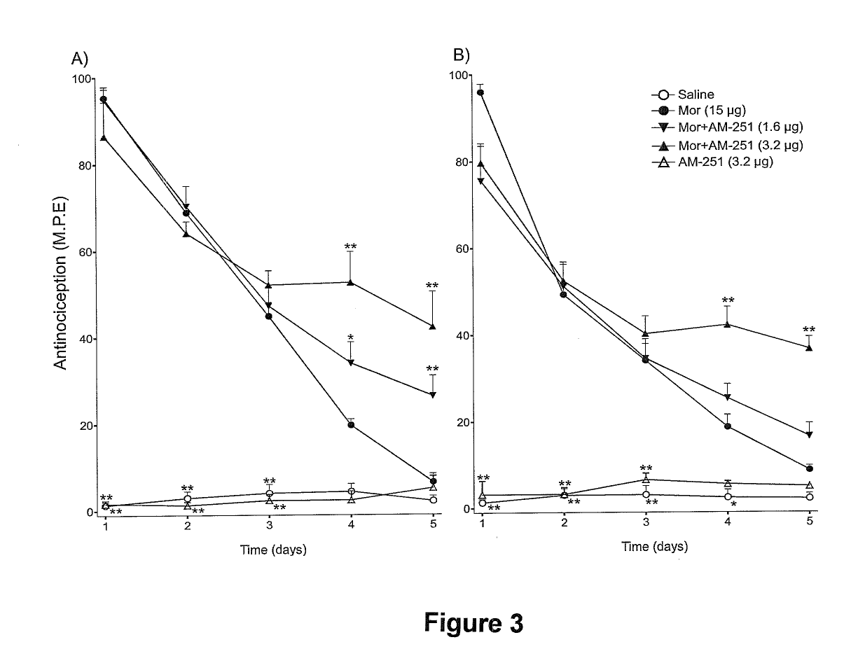

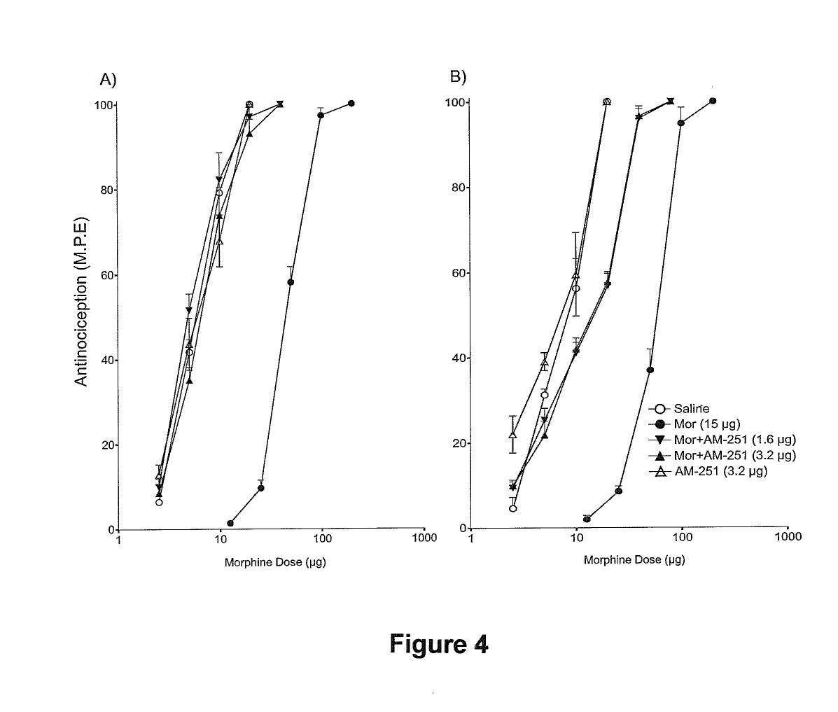

The effects of administration of a cannabinoid receptor antagonist on chronic morphine tolerance in rats were also examined (see FIGS. 3 and 4). FIG. 3 illustrates the effects of the cannabinoid receptor antagonist/inverse agonist AM-251 on the antinociceptive action of daily intrathecal morphine injection in the tail-flick (FIG. 3A) and paw-pressure (FIG. 3B) tests over 5-days. Intrathecal administration of morphine (15 μg; n=7) to naïve animals on day 1 produced maximal antinociception. However, repeated daily administration of morphine (15 μg) resulted in a progressive decline of antinociceptive effect to baseline levels comparable to saline-only control (n=4) by day 5. As measured by the tail-flick (FIG. 3A) and paw-pressure (FIG. 3B) nociceptive tests, co-administration of intrathecal AM-251 (3.2 μg; n=5) with morphine attenuated the development of tolerance. In particular, antinociception on day 4 and day 5 was significantly greater in the group co-treated with morphine and AM-251 as compared to the morphine-only treated group. Intrathecal injection of a lower dose of AM-251 (1.6 μg; n=8) attenuated the decline in morphine antinociception on day 4 (P<0.01) and day 5 (P<0.05) in the tail-flick test (FIG. 3A), but failed to have a significant effect in the paw-pressure test (FIG. 3B). When administered alone over 5 days, AM-251 (3.2 μg; n=4) did not have an effect on basal nociceptive responses.

Following 5-day chronic morphine treatment, cumulative dose-response curves were generated on day 6 to determine morphine analgesic potency in the tail-flick (FIG. 4A) and paw-pressure tests (FIG. 4B). Morphine administration in saline control (morphine naïve) animals produced a dose-dependent attenuation of the thermal and mechanical nociceptive response. Animals treated with 5-day intrathecal morphine (15 μg) required higher doses before maximal antinociception was achieved. This was reflected by a significant rightward shift in the cumulative dose-response curve and a 7.5-fold (P<0.01) and 5.9-fold (P<0.01) increase in ED₅₀ values in the tail-flick and paw-pressure tests, respectively, compared to saline-only controls (Table 1). Co-administration of AM-251 (1.6 μg or 3.2 μg) with intrathecal morphine for 5 days prevented the rightward shift in the cumulative dose-response curve and completely blocked the increase in ED₅₀ values (Table 1). ED₅₀ values obtained in groups given a combination of morphine and AM-251 (1.6 μg or 3.2 μg) were comparable to those in the saline control animals. Administration of AM-251 (3.2 μg) alone for 5 days did not significantly alter the morphine ED₅₀ value from that obtained with chronic saline treatment.

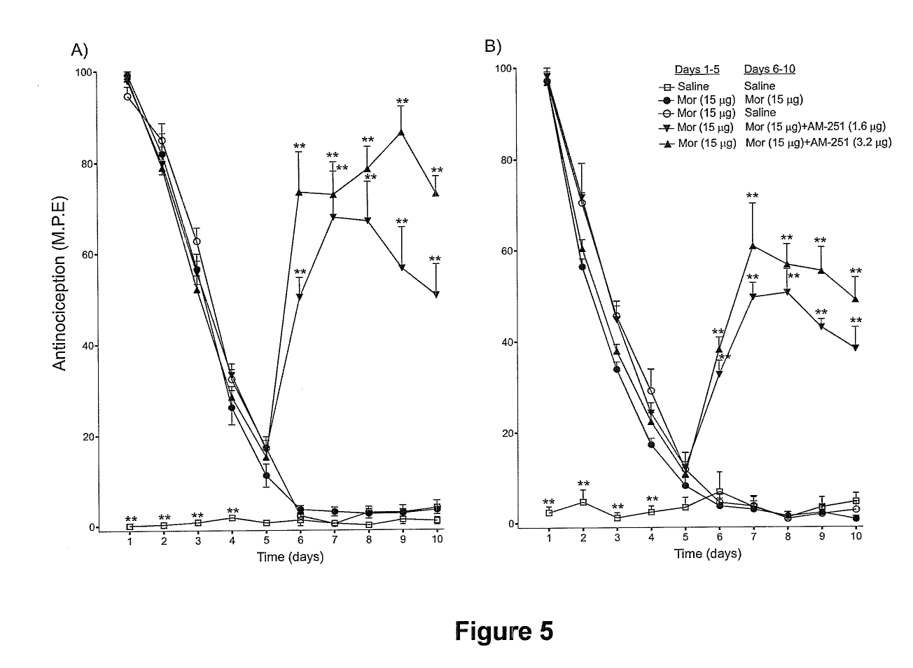

Maintenance of chronic morphine tolerance upon co-administration with a cannabinoid receptor antagonist was also examined. As shown in FIG. 5, daily intrathecal injection of morphine (3.2 μg; n=5) for 10 days produced a progressive decline in morphine antinociception to baseline levels by day 5, indicating the development of opioid analgesic tolerance. In opioid tolerant animals, co-administration of AM-251 (1.6 μg; n=5) on days 6-10 restored morphine antinociception to approximately 51% and 38% of the original analgesic response in the tail-flick (FIG. 5A) and paw-pressure (FIG. 5B) tests, respectively. At a higher dose, co-administration of AM-251 (3.2 μg; n=5) restored the morphine effect to approximately 73% and 50% of the original analgesic response in the tail-flick (FIG. 5A) and paw-pressure (FIG. 5B) tests, respectively. Intervention with AM-251 (3.2 μg; n=5) or saline in the absence of morphine on days 6-10 had no effect on basal nociceptive response. In addition, saline-only injection to morphine naïve animals for 10 days did not change baseline responses.

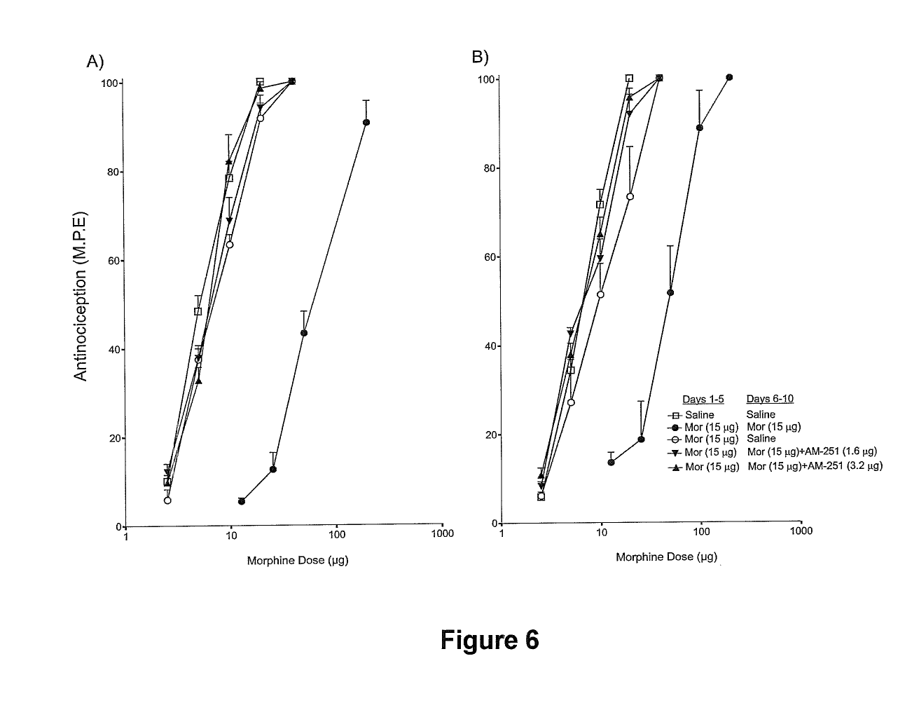

Following the 10 day chronic morphine treatment period, cumulative dose-response curves for the antinociceptive effect of intrathecal morphine was generated on day 11 (FIG. 6) and ED₅₀ values for morphine analgesia derived from the constructed dose-response curves (see Table 1). Acute morphine administration in saline-only (morphine-naïve) animals elicited a dose-dependent antinociceptive effect. Animals treated with 10 day intrathecal morphine (15 μg) required significantly higher doses of morphine before maximal antinociception was achieved. This was reflected by a rightward shift in the cumulative dose-response curve and a 9.6-fold (p<0.01) increase in morphine ED₅₀ value in the tail-flick test (FIG. 6A) and a 6.8-fold (p<0.01) increase in the paw-pressure test (FIG. 6B) compared to saline-only (morphine-naïve) animals. The increase in morphine ED₅₀ value thus reflects a substantial loss of the opioid analgesic potency. In animals already rendered opioid tolerant, co-administration of morphine and AM-251 (1.6 or 3.2 μg) on days 6-10 reversed the increase in morphine ED₅₀ value indicating restoration of the opioid drug potency. ED₅₀ values obtained in groups given morphine and AM-251 (1.6 or 3.2 μg) were comparable to those in the saline-only (morphine-naïve) animals. Discontinuation of intrathecal morphine and the subsequent injection of saline, or AM-251 alone, on days 6-10 resulted in ED₅₀ value comparable to saline-only (morphine naïve) animals, which suggest that abstinence from morphine for 5 days is sufficient to restore morphine analgesic potency in opioid tolerant animals.

TABLE 1
Effect of intrathecal AM-251 on spinal morphine
tolerance

| | |
|---|---|
| Tail-flick | Paw-Pressure |
| ED₅₀ (μg) | ED₅₀ (μg) |
| (mean ± | (mean ± |
| s.e.mean) | s.e.mean) |

Acute Tolerance

| | | |
|---|---|---|
| Saline | 4.9 ± 0.3 | 5.3 ± 0.7 |
| Mor (15 μg), then Saline only | 6.5 ± 0.1 | 8.9 ± 0.4 |
| Mor (15 μg) | 33.7 ± 0.9** | 35.2 ± 3.3** |
| Mor (15 μg) + AM-251 (1.6 μg) | 3.3 ± 0.2 | 4.9 ± 0.7 |
| Mor (15 μg) + AM-251 (3.2 μg) | 3.3 ± 0.5 | 5.6 ± 0.8 |
| AM-251 (3.2 μg) | 5.6 ± 0.1 | 4.6 ± 0.6 |

Chronic Tolerance

| | | |
|---|---|---|
| Days 1-5 | | |
| Saline | 5.5 ± 0.1 | 8.7 ± 1.6 |
| Mor (15 μg) | 46.7 ± 2.1** | 59.9 ± 4.0** |
| Mor (15 μg) + AM-251 (1.6 μg) | 5.0 ± 0.5 | 12.4 ± 0.9 |
| Mor (15 μg) + AM-251 (3.2 μg) | 6.5 ± 0.5 | 12.5 ± 0.8 |
| AM-251 (3.2 μg) | 6.8 ± 0.7 | 13.7 ± 0.8 |

Maintenance of Chronic Tolerance

| | | | |
|---|---|---|---|
| Days 1-5 | Days 6-10 | | |
| Saline | Saline | 5.0 ± 0.3 | 6.4 ± 1.3 |
| Mor (15 μg) | Saline | 6.8 ± 0.4 | 10.7 ± 2.2 |
| Mor (15 μg) | Mor (15 μg) | 53.0 ± 3.6** | 49.8 ± 6.5** |
| Mor (15 μg) | Mor + AM-251 (1.6 μg) | 6.1 ± 0.6 | 6.7 ± 0.5 |
| Mor (15 μg) | Mor + AM-251 (3.2 μg) | 5.7 ± 0.5 | 7.0 ± 0.6 |
| Mor (15 μg) | AM-251 (3.2 μg) | 6.0 ± 0.2 | 6.2 ± 0.3 |

ED₅₀ values were derived from cumulative dose-response curves to acute intrathecal morphine generated after the morphine treatment period. In the acute morphine tolerance paradigm, three successive intrathecal morphine injections were administered over 4 hours and ED₅₀ values determined the following day. In the chronic morphine tolerance paradigm, intrathecal morphine was given daily over 5 days and ED₅₀ values were generated on day 6. In the maintenance of the chronic tolerance paradigm, animals were given intrathecal morphine for 10 days and ED₅₀ values determined on day 11. * is representative of a significant difference from saline-treated control group; **(P<0.01).

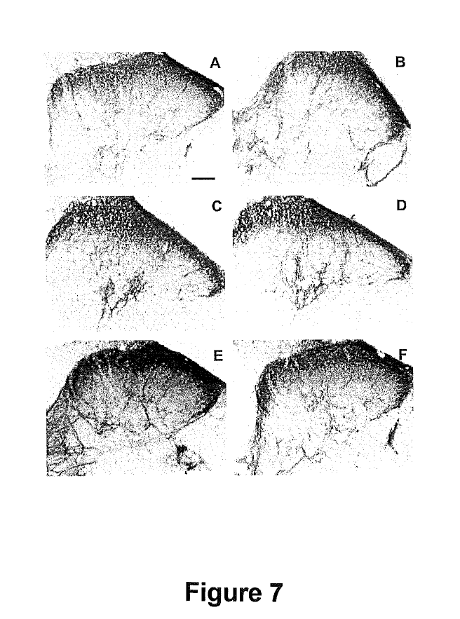

Experiments were also performed to examine the effect of AM-251 on morphine-induced changes in spinal CGRP-immunoreactivity. Representative photomicrographs of CGRP-immunoreactive neurons are shown in FIG. 7. The corresponding semi-quantitative data from measurements of mean optical density in the L4-L5 dorsal horn region of rats given intrathecal drug treatment is represented in Table 2. In saline treated animals, CGRP-immunoreactivity was localized primarily in the superficial laminae of the dorsal horn (FIG. 7A). In this region, daily intrathecal morphine treatment for 5-days produced a 46% (P<0.001) increase in the mean optical density of CGRP-immunoreactivity (FIG. 7C); however, no significant effect on CGRP-immunoreactivity in the deeper laminae of the spinal dorsal horn was detected. The morphine-induced increase in CGRP-immunoreactivity was suppressed by co-treatment of morphine with AM-251 (3.2 μg) (FIG. 7D). Treatment with AM-251 (3.2 μg) in the absence of morphine for 5-days had no noticeable effect on CGRP-immunoreactivity.

In contrast to the localized increase in CGRP-immunoreactivity in the superficial laminae induced by 5 day morphine treatment, intrathecal injection of morphine (15 μg) for 10 days increased CGRP expression throughout the entire dorsal horn region of the spinal cord (FIG. 7E). Notably, the mean optical density of CGRP-immunoreactivity in 10 day morphine-treated animals was increased by 38% (P<0.01) and 45% (P<0.01) in the superficial and deeper laminae, respectively, as compared to saline-treated controls. In animals rendered morphine tolerant, co-administration of morphine with AM-251 (3.2 μg) on days 6-10 partially reversed the increase in CGRP-immunoreactivity (FIG. 7F). Specifically, the mean optical density of CGRP in this group was reduced by 12% (P<0.05) and 9% (P<0.05) in the superficial and deeper laminae, respectively, as compared to the 10 day morphine-only treated group. Cessation of morphine treatment after day 5 and the subsequent injection of only saline on days 6-10 resulted in the return of CGRP-immunoreactivity to baseline levels (Table 2). The effect of AM-251 (3.2 μg) alone had no effect on basal CGRP-immunoreactivity as compared to saline treated (morphine naïve) control (Table 2).

In contrast to the 5 and 10 day chronic morphine treatment paradigms, acute intrathecal injection of morphine (15 μg) over 4 hours leading to the rapid induction of acute analgesic tolerance failed to have an effect on CGRP-immunoreactivity (FIG. 7B). In fact, optical density measurements revealed that CGRP-immunoreactivity in the spinal dorsal horn of acutely tolerant animals was not different from that of saline-only treated (morphine naïve) controls (Table 2). Indeed, examination of optical density values indicated that treatment with AM-251 (1.6 or 3.2 μg) in the absence of morphine had no effect on basal CGRP expression (Table 2).

TABLE 2
Effect of intrathecal AM-251 on the mean optical
density (OD) of CGRP-immunoreactivity in the spinal dorsal
horn

| | | | |
|---|---|---|---|
| Superficial | | Deeper | |
| Laminae | Relative | Laminae | Relative |
| (mean ± s.e.mean) | OD | (mean ± s.e.mean) | OD |

Acute Tolerance

| | | | | |
|---|---|---|---|---|
| Saline | 136.8 ± 5.7 | 1.00 | 73.9 ± 10.0 | 1.00 |
| Mor | 146.0 ± 6.7 | 1.06 | 81.6 ± 7.8 | 1.10 |
| Mor + AM-251 | 131.7 ± 7.0 | 0.96 | 80.4 ± 8.5 | 1.09 |
| AM-251 | 142.4 ± 4.2 | 1.04 | 67.2 ± 4.1 | 0.91 |

Chronic
Tolerance

| | | | | |
|---|---|---|---|---|
| Days 1-5 | | | | |
| Saline | 133.9 ± 1.7 | 1.00 | 70.3 ± 12.2 | 1.00 |
| Mor | 195.7 ± 12.0** | 1.46** | 89.5 ± 11.9 | 1.27 |
| Mor + AM-251 | 140.2 ± 7.1 | 1.05 | 59.8 ± 16.8 | 0.94 |
| AM-251 | 139.9 ± 3.6 | 1.04 | 60.4 ± 8.0 | 0.95 |

Maintenance
of Chronic
Tolerance

| | | | | | |
|---|---|---|---|---|---|
| D1-5 | D6-10 | | | | |
| Saline | Saline | 127.7 ± 6.8 | 1.00 | 70.9 ± 7.0 | 1.00 |
| Mor | Saline | 141.8 ± 4.5 | 1.11 | 84.8 ± 13.6 | 1.20 |
| Mor | Mor | 176.1 ± 8.2* | 1.38* | 103.0 ± 7.5* | 1.45* |
| Mor | Mor + AM-251 | 155.0 ± 6.3 | 1.21 | 93.3 ± 14.1 | 1.32 |
| Mor | AM-251 | 131.9 ± 7.6 | 1.03 | 77.1 ± 2.3 | 1.09 |

Morphine was administered at 15 μg and AM-251 was administered at 3.2 μg.
*Represents significant difference from saline-treated control group;
*(P < 0.01);
**(P < 0.001).

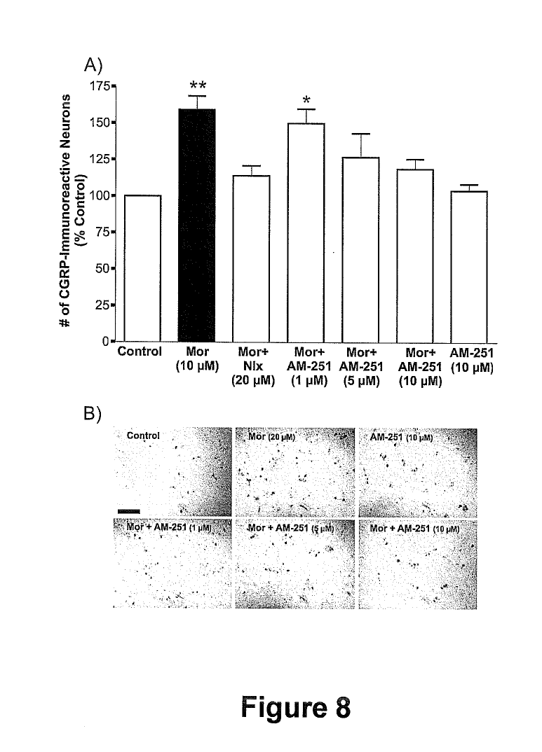

The effects of AM-251 on morphine-induced changes in CGRP-immunoreactivity in cultured dorsal root ganglion neurons were also examined. For these experiments, dorsal root ganglion (DRG) cultures were prepared and maintained for one week prior to CGRP immunostaining. Representative photomicrographs and quantification of the number of CGRP-immunopositive DRG neurons are represented in FIG. 8. Chronic morphine treatment for 5-days increased the number of CGRP-immunopositive neurons by approximately 57% (P<0.01) as compared to vehicle-treated controls. This response was blocked by pre-treatment with naloxone (10 μM), a non-selective opioid receptor antagonist, indicating that the increase in CGRP-immunoreactivity is indeed mediated by opioid receptor activity. To investigate the potential role of endocannabinoids in the morphine-induced increase in CGRP-immunoreactive neurons, AM-251 was co-administered with morphine for 5 days in the DRG culture. Co-treatment of AM-251 at 10 μM concentration suppressed the morphine-induced increase in CGRP-immunoreactivity by 25% (P<0.05). At 5 μM concentration, AM-251 decreased the number of CGRP-immunoreactive neurons by 20% as compared to morphine-only treated group; however, this did not achieve statistical significance. No effect on the morphine-induced increase in CGRP-immunoreactivity was seen following co-treatment of morphine with 1 μM of AM-251. AM-251 (10 μM) treatment in the absence of morphine had no effect on basal CGRP expression in DRG neurons.

Thus, as shown by these experiments, coupling repeated administration of intrathecal morphine with AM-251, a potent and selective CB₁-receptor antagonist/inverse agonist, prevents both the decline in level of analgesia and loss of opioid agonist potency. At the biochemical level, this coupling prevents the morphine-induced increase in CGRP-immunoreactivity in the dorsal horn and in the cultured adult DRG neurons, suggesting that its locus of action is at the level of sensory neurons. Further, when co-administered with morphine to chronic tolerant animals, AM-251 partially restored the analgesic actions of morphine and reversed the increase in spinal CGRP-immunoreactivity. Thus, CB₁-receptor activity not only modulates responses associated with opioid withdrawal (Trang et al. Pain 2006 126(1-3):256-71) but it also influences responses signaling the analgesic tolerance that is associated with increased expression of CGRP in sensory neurons.

Although opioid tolerance is generally recognized to follow chronic drug exposure, it can also occur upon repeated acute opioid exposure (Fairbanks, C. A. and Wilcox, G. L., J. Pharmacol. Exp. Ther. 1997 282:1408-1417). Animal studies have revealed that both states share a common underlying mechanism involving activation of spinal NMDA receptors (Fairbanks, C. A. and Wilcox, G. L., J. Pharmacol. Exp. Ther. 1997 282:1408-1417). As shown herein, repeated injection of spinal morphine over a 4 hour period not only produced a profound loss of analgesia but also a highly significant reduction of agonist potency, as indicated by increased morphine ED₅₀ values reflective of the development of acute tolerance. Importantly, these indices of tolerance were apparent in both nociceptive tests and were comparable to those obtained after 5-day chronic morphine treatment. In both dosing paradigms, co-treatment with AM-251 exerted similar behavioral effects, suggesting a common cannabinoid-receptor based mechanism in the induction of acute and chronic opioid tolerance. AM-251 also reversed established chronic tolerance, indicating that activity of cannabinoid-receptors contributes to the maintenance of this phenomenon.

The dose of the cannabinoid receptor antagonist AM-251 demonstrated to be effective in attenuating development of opioid tolerance herein was previously shown to selectively block the anandamide-induced antinociception but have no influence on acute morphine-induced analgesia (Trang et al. Pain 2006 126(1-3):256-71). Additionally, in the present study intrathecal injection of AM-251 alone did not change nociceptive responses and had no effect on behavioral or biochemical indices of opioid tolerance. Thus, the attenuation of opioid tolerance was not the result of an AM-251-mediated shift in basal CB₁-receptor activity nor was it simply a direct influence of AM-251 on the acute analgesic response to morphine.

As will be understood by the skilled artisan upon reading this disclosure, the present invention is not limited to the specific examples of potentiating therapeutic activity and/or inhibiting, delaying, reducing and/or reversing tolerance set forth herein, but rather, the invention should be construed and understood to include any combination of an opioid receptor agonist and cannabinoid receptor antagonist wherein such combination has the ability to potentiate a therapeutic activity of an opioid receptor agonist and/or inhibit, delay, reduce and/or reverse tolerance to an opioid receptor agonist therapy. Based on the teachings set forth in extensive detail elsewhere herein, the skilled artisan will understand how to identify such opioid receptor agonists, cannabinoid receptor antagonists, and combinations thereof, as well as the concentrations of opioid receptor agonists and cannabinoid receptor antagonists to use in such a combination useful in the present invention.

As demonstrated herein, opioid receptor agonists and cannabinoid receptor antagonists can be administered, for example, epidurally or intrathecally. Further, as many of these compounds including morphine are known to be effective by systemic administration, i.e. orally or parenterally, it is expected that these therapeutic compounds will be effective following systemic administration as well. Accordingly, the combination therapies of the invention may be administered systemically or locally, and by any suitable route such as oral, buccal, sublingual, transdermal, subcutaneous, intraocular, intravenous, intramuscular or intraperitoneal administration, and the like (e.g., by injection) or via inhalation. Preferably, the opioid receptor agonist and cannabinoid receptor antagonist are administered simultaneously via the same route of administration. However, it is expected that administration of the compounds separately, via the same route or different route of administration, within a time frame during which each therapeutic compound remains active, will also be effective in pain management as well as in inhibiting, delaying, reducing and/or reversing tolerance to the opioid receptor agonist. Further, administration of a cannabinoid receptor antagonist to a subject already receiving opioid receptor agonist treatment is expected to reverse any tolerance to the opioid receptor agonist and restore analgesic potency of the opioid receptor agonist. Thus, treatment with the opioid receptor agonist and cannabinoid receptor antagonist in the combination therapy of the present invention need not begin at the same time. Instead, administration of the cannabinoid receptor antagonist may begin several days, weeks, months or more after treatment with the opioid receptor agonist. Alternatively, administration of the cannabinoid receptor antagonist may begin several days, weeks, months or more before treatment with the opioid receptor agonist.

Accordingly, for purposes of the present invention, the therapeutic compounds, namely the opioid receptor agonist and the cannabinoid receptor antagonist, can be administered together in a single pharmaceutically acceptable vehicle or separately, each in their own pharmaceutically acceptable vehicle.

As used herein, the term “therapeutic compound” is meant to refer to an opioid receptor agonist and/or a cannabinoid receptor antagonist.

As used herein “pharmaceutically acceptable vehicle” includes any and all solvents, excipients, dispersion media, coatings, antibacterial and antifungal agents, isotonic and absorption delaying agents, and the like which are compatible with the activity of the therapeutic compound and are physiologically acceptable to a subject. An example of a pharmaceutically acceptable vehicle is buffered normal saline (0.15 M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

Carrier or substituent moieties useful in the present invention may also include moieties which allow the therapeutic compound to be selectively delivered to a target organ. For example, delivery of the therapeutic compound to the brain may be enhanced by a carrier moiety using either active or passive transport (a “targeting moiety”). Illustratively, the carrier molecule may be a redox moiety, as described in, for example, U.S. Pat. Nos. 4,540,654 and 5,389,623, both to Bodor. These patents disclose drugs linked to dihydropyridine moieties which can enter the brain, where they are oxidized to a charged pyridinium species which is trapped in the brain. Thus drugs linked to these moieties accumulate in the brain. Other carrier moieties include compounds, such as amino acids or thyroxine, which can be passively or actively transported in vivo. Such a carrier moiety can be metabolically removed in vivo, or can remain intact as part of an active compound.

Structural mimics of amino acids (and other actively transported moieties) including peptidomimetics, are also useful in the invention. As used herein, the term “peptidomimetic” is intended to include peptide analogues which serve as appropriate substitutes for peptides in interactions with, for example, receptors and enzymes. The peptidomimetic must possess not only affinity, but also efficacy and substrate function. That is, a peptidomimetic exhibits functions of a peptide, without restriction of structure to amino acid constituents. Peptidomimetics, methods for their preparation and use are described in Morgan et al. (1989) (“Approaches to the discovery of non-peptide ligands for peptide receptors and peptidases,” In Annual Reports in Medicinal Chemistry (Virick, F. J., ed.), Academic Press, San Diego, Calif., pp. 243-253), the contents of which are incorporated herein by reference. Many targeting moieties are known, and include, for example, asialoglycoproteins (see e.g., Wu, U.S. Pat. No. 5,166,320) and other ligands which are transported into cells via receptor-mediated endocytosis (see below for further examples of targeting moieties which may be covalently or non-covalently bound to a target molecule).

The term “subject” as used herein is intended to include living organisms in which pain to be treated can occur. Examples of subjects include mammals such as humans, apes, monkeys, cows, sheep, goats, dogs, cats, mice, rats, and transgenic species thereof. As would be apparent to a person of skill in the art, the animal subjects employed in the working examples set forth below are reasonable models for human subjects with respect to the tissues and biochemical pathways in question, and consequently the methods, therapeutic compounds and pharmaceutical compositions directed to same. As evidenced by Mordenti (J. Pharm. Sci. 1986 75(11):1028-40) and similar articles, dosage forms for animals such as, for example, rats can be and are widely used directly to establish dosage levels in therapeutic applications in higher mammals, including humans. In particular, the biochemical cascade initiated by many physiological processes and conditions is generally accepted to be identical in mammalian species (see, e.g., Mattson and Scheff, Neurotrauma 1994 11(1):3-33; Higashi et al. Neuropathol. Appl. Neurobiol. 1995 21:480-483). In light of this, pharmacological agents that are efficacious in animal models such as those described herein are believed to be predictive of clinical efficacy in humans, after appropriate adjustment of dosage.

Depending on the route of administration, the therapeutic compound may be coated in a material to protect the compound from the action of acids, enzymes and other natural conditions which may inactivate the compound. Insofar as the invention provides a combination therapy in which two therapeutic compounds are administered, each of the two compounds may be administered by the same route or by a different route. Also, the compounds may be administered either at the same time (i.e., simultaneously) or each at different times. In some treatment regimes it may be beneficial to administer one of the compounds more or less frequently than the other.

The compounds of the invention can be formulated to ensure proper distribution in vivo. For example, the blood-brain barrier (BBB) excludes many highly hydrophilic compounds. To ensure that the therapeutic compounds of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., Ranade, V. V. J. Clin. Pharmacol. 1989 29(8):685-94). Exemplary targeting moieties include folate and biotin (see, e.g., U.S. Pat. No. 5,416,016 to Low et al.); mannosides (Umezawa et al. Biochem. Biophys. Res. Commun. 1988 153(3):1038-44; antibodies (Bloeman et al. FEBS Lett. 1995 357:140; Owais et al. Antimicrob. Agents Chemother. 1995 39(1):180-4); and surfactant protein A receptor (Briscoe et al. Am. J. Physiol. 1995 268 (3 Pt 1):L374-80). In a preferred embodiment, the therapeutic compounds of the invention are formulated in liposomes; in a more preferred embodiment, the liposomes include a targeting moiety.

Delivery and in vivo distribution can also be affected by alteration of an anionic group of compounds of the invention. For example, anionic groups such as phosphonate or carboxylate can be esterified to provide compounds with desirable pharmacokinetic, pharmacodynamic, biodistributive, or other properties.

To administer a therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. Prog. Clin. Biol. Res. 1984 146:429-34).

The therapeutic compound may also be administered parenterally (e.g., intramuscularly, intravenously, intraperitoneally, intraspinally, intrathecally, or intracerebrally). Dispersions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g., vegetable oil). The proper fluidity can be maintained, for example, by the use of a coating such as lecithin, by the maintenance of the required particle size in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In some cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, wafers, buccal tablets, troches, and the like. In such solid dosage forms the active compound is mixed with at least one inert, pharmaceutically acceptable excipient or diluent or assimilable edible carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compound in the compositions and preparations may, of course, be varied. The amount of the therapeutic compound in such therapeutically useful compositions is such that a suitable dosage will be obtained.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Therapeutic compounds can be administered in time-release or depot form, to obtain sustained release of the therapeutic compounds over time. The therapeutic compounds of the invention can also be administered transdermally (e.g., by providing the therapeutic compound, with a suitable carrier, in patch form).

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of neurological conditions in subjects.

Therapeutic compounds according to the invention are administered at a therapeutically effective dosage sufficient to achieve the desired therapeutic effect of the opioid receptor agonist, e.g. to mitigate pain and/or to effect analgesia in a subject, to suppress coughs, to reduce and/or prevent diarrhea, to treat pulmonary edema or to alleviate addiction to opioid receptor agonists. For example, if the desired therapeutic effect is analgesia, the “therapeutically effective dosage” mitigates pain by about 25%, preferably by about 50%, even more preferably by about 75%, and still more preferably by about 100% relative to untreated subjects. Actual dosage levels of active ingredients in the pharmaceutical compositions of this invention may be varied so as to obtain an amount of the active compound(s) that is effective to achieve and maintain the desired therapeutic response for a particular subject, composition, and mode of administration. The selected dosage level will depend upon the activity of the particular compound, the route of administration, frequency of administration, the severity of the condition being treated, the condition and prior medical history of the subject being treated, the age, sex, weight and genetic profile of the subject, and the ability of the therapeutic compound to produce the desired therapeutic effect in the subject. Dosage regimens can be adjusted to provide the optimum therapeutic response. For example, several divided doses may be administered daily or the dose may be proportionally reduced as indicated by the exigencies of the therapeutic situation.

However, it is well known within the medical art to determine the proper dose for a particular patient by the dose titration method. In this method, the patient is started with a dose of the drug compound at a level lower than that required to achieve the desired therapeutic effect. The dose is then gradually increased until the desired effect is achieved. Starting dosage levels for an already commercially available therapeutic agent of the classes discussed above can be derived from the information already available on the dosages employed. Also, dosages are routinely determined through preclinical ADME toxicology studies and subsequent clinical trials as required by the FDA or equivalent agency. The ability of an opioid receptor agonist to produce the desired therapeutic effect may be demonstrated in various well known models for the various conditions treated with these therapeutic compounds. For example, mitigation of pain can be evaluated in model systems that may be predictive of efficacy in mitigating pain in human diseases and trauma, such as animal model systems known in the art (including, e.g., the models described herein).

Compounds of the invention may be formulated in such a way as to reduce the potential for abuse of the compound. For example, a compound may be combined with one or more other agents that prevent or complicate separation of the compound therefrom.

The following nonlimiting examples are provided to further illustrate the present invention. The contents of all references, pending patent applications, and published patents cited throughout this application are hereby expressly incorporated by reference.

### EXAMPLES

### Example 1

### Animals

Experiments were performed in accordance with guidelines of the Canadian Council on Animal Care using protocols approved by the University Animal Care Committee. Adult male Sprague Dawley rats (200-250 grams) (Charles River, Quebec) were housed in separate cages and maintained on a 12 hour light/12 hour dark cycle with access to food and water ad libitum.

### Example 2

### Intrathecal Catheterization

Animals were anaesthetized with halothane (4%) and implanted with an indwelling intrathecal catheter. A small puncture was made in the atlanto-occipital membrane and a polythene catheter (PE-10; 7.5 cm long) inserted caudally so that the tip rested on the lumbar enlargement of the spinal cord. The rostral end was exteriorized to facilitate drug administration. Surgical wounds were closed with sutures and animals allowed to recover for one week. Animals showing signs of forelimb or hindlimb paralysis were excluded from experiments. Drugs were injected in a 10 μl volume (i.t.) followed by 10 μl of 0.9% saline to flush the catheter.

### Example 3

### Behavioural Assessment of Nociception

Tail-Flick Test

Response to an acute thermal nociceptive stimulus was assessed in rats using the tail-flick test. Radiant heat was applied to the dorsal surface of the tail using an analgesic meter and the time latency for removal of the tail from the stimulus was recorded. The heat source was adjusted to yield a baseline response of 2-3 seconds, which allowed measurement of morphine-induced antinociception. A cutoff of 10 seconds was used in both studies to prevent tissue injury.

Paw-Pressure Test

Response to an acute mechanical nociceptive stimulus was assessed using the paw-pressure test. Using an air-filled inverted syringe, pressure was gradually applied to the dorsal surface of the hindpaw until a paw withdrawal response was observed and the value (mmHg) recorded. A cut-off of 300 mmHg was used to prevent tissue injury.

### Example 4

### Behavioral Effects of AM-251 on Acute and Chronic Morphine Tolerance

Acute Morphine Tolerance

Animals were rendered acutely tolerant to the analgesic effects of morphine using three successive intrathecal injections of morphine (15 μg) administered at 0 minutes, 90 minutes, and 180 minutes. Tail-flick and paw-pressure nociceptive testing was performed prior to morphine administration to determine baseline response. After morphine injection, nociceptive testing was performed at 30 minute intervals over a 4 hour testing period. To examine the effects of AM-251 (1.6 or 3.2 μg) on acute tolerance, the antagonist/inverse agonist was co-administered with intrathecal morphine (15 μg) at the 0 minute, 90 minute, and 180 minute injection time-points and the effects of AM-251 on morphine analgesia assessed at 30 minute intervals.

Twenty four hours following induction of acute tolerance, a cumulative dose-response curve was generated to determine morphine potency. To assess changes in analgesic potency, animals were given ascending doses of intrathecal morphine every 30 minutes until a maximal level of antinociception was reached in both the tail-flick and paw-pressure test. Morphine ED₅₀ value, an indicator of drug potency, was derived from the cumulative dose-response curve. A progressive decline in the daily antinociceptive effect and an increase in the ED₅₀ value indicate a loss in morphine potency and reflect a state of analgesic tolerance. The ability of AM-251 to prevent induction of acute morphine tolerance was assessed by examining its effect on the decline in morphine antinociception and on changes in morphine potency. One day following determination of morphine potency, spinal cords from animals were processed for CGRP immunohistochemical staining as described infra.

Chronic Morphine Tolerance

To induce chronic tolerance, animals were given an intrathecal injection of morphine (15 μg) once daily between 10 and 11 AM for 5 days, in accordance with procedures described by Powell et al. (Br. J. Pharmacol. 2000 131:875-884). Tail-flick and paw-pressure nociceptive tests were performed both before and 30 minutes after drug injection. The peak antinociceptive effect of intrathecal morphine has been shown to occur 30 minutes post-injection (Powell et al., Br. J. Pharmacol. 2000 131:875-884). To determine the effect of AM-251 (1.6 or 3.2 μg) on the development of tolerance to chronic morphine, the antagonist was intrathecally co-administered with morphine for 5 days. Nociceptive testing was performed both before and 30 minutes after each injection. On day 6, animals were given ascending doses of morphine every 30 minutes until a maximal level of antinociception was reached in both the tail-flick and paw-pressure test and morphine ED₅₀ value determined. The ability of AM-251 to prevent the development of tolerance to chronic morphine was assessed by examining its effect on the decline in morphine antinociception and on changes in morphine potency. One day following determination of morphine potency, spinal cords from animals were processed for CGRP immunohistochemical staining as described infra.

Maintenance of Chronic Morphine Tolerance

To determine the effects of AM-251 on established morphine tolerance, animals were first rendered tolerant using once daily intrathecal injection of morphine (15 μg) for 5 days as described supra. On days 6 to 10, AM-251 (1.6 or 3.2 μg) was administered either in combination with morphine or alone (control). Morphine ED₅₀ values were determined on day 11 and cumulative dose-response curves generated as described supra. The ability of AM-251 to reverse established morphine tolerance was reflected by recovery of both morphine antinociception and potency. The day following ED₅₀ testing, spinal cords from animals were processed for CGRP immunohistochemical staining as described infra.

### Example 5

### Effect of AM-251 on Morphine-Induced Changes in CGRP-Immunoreactivity in Spinal Dorsal Horn Neurons

Animals were anaesthetized with urethane and perfused intracardially with 0.1 M phosphate-buffered 4% paraformaldehyde in accordance with procedures described by Powell et al. (Br. J. Pharmacol. 2000 131:875-884). The lumbar segment of the spinal cord was dissected, post-fixed overnight in 4% paraformaldehyde, and then transferred to 30% sucrose. The spinal cord was sectioned at 40 μm thickness using a cryostat. Immunohistochemical staining of spinal cord sections were performed free-floating followed by incubation with 0.3% H₂O₂ for 30 minutes prior to incubation with 10% normal goat serum (NGS) for 1 hour. Sections were then incubated with rabbit polyclonal anti-CGRP antibody (1:4000; Chemicon International, Temecula, Calif.) diluted in phosphate buffered saline containing 0.3% Triton X-100 (PBS-T) for 36 hours at 4° C. Following 1 hour incubation with biotinylated anti-rabbit secondary antibody (1:200; Vector Laboratories Inc., Burlingame, Calif.), sections were processed with Vecastain ABC kit (Vector Laboratories Inc.) and developed using 3,-3 diaminobenzedine (Vector Laboratories Inc.). To minimize variation in staining densities spinal tissue from all groups were immunostained simultaneously.

CGRP-like immunoreactivity in spinal cord sections was quantified by measuring relative optical density using image analysis software (Imaging Research Inc., St. Catherine, ON, Canada). Five spinal cord sections were randomly taken from five rats in each of the treatment groups outlined above. Optical density measurements were taken from 2 regions of the spinal dorsal horn: the superficial laminae (I-II) and deeper laminae (III-VI). Optical density measurements for CGRP-like immunoreactivity in the spinal dorsal horn region of all treatment groups were compared to the morphine-only treated group to determine the effects of drug treatment on CGRP expression following morphine exposure. The density readings were performed using identical background intensity settings and compared between treatment groups to measure relative changes. Images of the dorsal horn regions were taken at 10× magnification using a high-resolution CCD camera.

### Example 6

### Effect of AM-251 on Morphine-Induced Changes in CGRP-Immunoreactivity in Cultured Dorsal Root Ganglion Neurons

Primary Dorsal Root Ganglion Cultures

Dorsal root ganglions (DRG) were isolated from adult male Sprague Dawley rats (200-250 grams) according to the method of Ma et al. (Neuroscience 2000 99:529-539) and by Powell et al. (Eur. J. Neurosci. 2003 18:1572-1583). In this method, rats were decapitated and DRGs removed aseptically from the cervical, thoracic, lumbar, and sacral regions of the spinal cord. DRGs were collected in modified Hank's Balanced Salt Solution (HBSS, GIBCO/BRL, Burlington, ON, Canada) containing 1% HEPES buffer (pKa 7.55, GIBCO/BRL) and penicillin/streptomycin (1:1000, GIBCO/BRL). DRGs were minced, digested with 0.25% collagenase (Calbiochem) in Ham's F12 Medium (GIBCO/BRL) for 90 minutes at 37° C., and then trypsinized (0.25%) (GIBCO/BRL) for 1.5 hours at 37° C. Next, the tissues were triturated with a 19 gauge syringe in Dulbecco's Modified Eagle Medium (DMEM, GIBCO/BRL) containing 1% HEPES buffer, penicillin/streptomycin (1:100), and 10% Fetal Bovine Serum (GIBCO/BRL). Samples were then centrifuged at 500 g for 10 minutes. The resulting pellet was resuspended with DMEM and filtered through a cell strainer (70 μm, Falcon). DRG cells were seeded in a poly-D-lysine coated 96-well culture plate (Falcon) at a density of 5×10⁴ cells/well and incubated at 37° C. with 5% CO₂ and 95% O₂ for the duration of the experiment.

Two days later cells were plated, the culture medium was changed and drug treatment commenced. Drugs were prepared in culture medium and added to the cultures every other day for 5 days. Drug treatment consisted of either morphine (20 μM) or morphine in combination with AM-251 (1, 5 or 10 μM). In control wells, only culture medium was added (vehicle treatment), while positive control groups were tested with morphine (20 μM)+naloxone (10 μM), or AM-251 (10 μM) in the absence of morphine. Each drug treatment was repeated in at least 4 different wells of each culture and performed in at least four separate experiments. For each experiment, new primary cultures were prepared under standardized experimental conditions as described above.

CGRP-Immunohistochemistry in Cultured Dorsal Root Ganglion Neurons

After 5 days of drug treatment, DRG cells were fixed in 4% paraformaldehyde in 0.1M phosphate buffer for 20-25 minutes. The cells were pre-treated with 0.3% H₂O₂ and 10% normal goat serum (NGS, Vector Laboratories, Burlingame, Calif., USA.) in 0.01M phosphate buffered saline containing 0.3% Triton-X 100 (PBS+T) and then incubated with polyclonal rabbit antibodies raised against human CGRP (1:4000, Peninsula, Belmont, Calif., USA) for 48 hours at 4° C. Next, the cells were incubated in biotinylated goat anti-rabbit IgG and processed using an Elite Vectastain ABC kit (Vector Laboratories). The resulting immunoprecipitate was visualized using the glucose oxidase-nickel-3,-3′ diaminobenzedine method. The cells were maintained in 70% glycerol (Sigma Chemical Co.).

### Quantification of CGRP-Immunoreactivity in Cultured Dorsal Root Ganglion Neurons

To examine the effects of drug treatment on CGRP-immunoreactivity in the cultured DRG cells, the number of CGRP immunopositive cells was counted using an inverted phase microscope (Olympus CX2). An objective of ×10 was used, producing a final magnification of ×100. The field of view in which CGRP-IR cells were counted measured 1 mm². Ten fields of view were randomly chosen in each well to determine the average number of immunopositive cells. The average number of immunopositive cells per treatment group was obtained from four different wells in a single culture. Cell counts were obtained from a minimum of four separate cell cultures for each treatment group. Representative photomicrographs of the stained cells were taken using a Nikon Coolpix digital camera.

### Example 7

### Drugs

Morphine sulphate (BDH Pharmaceuticals, Canada) and naloxone (Sigma, USA) were dissolved in saline. AM-251 (1-(2,4-dichlorophenyl)-5-(4-iodophenyl)-4-methyl-N-1-piperidinyl-1H-pyrazole-3-carboxamide) (Tocris-Cookson, USA) was dissolved in dimethyl sulfoxide (DMSO) and diluted with saline (0.9%) to make a final concentration containing 1% DMSO. For experiments in DRG cultures, stock concentration of each drug was prepared as described and diluted to the final working concentration with culture media.

### Example 8

### Data Analysis

Tail-flick and paw-pressure values were converted to a maximum percentage effect (M.P.E.): M.P.E.=100×[post-drug response−baseline response]/[maximum response−baseline response]. Data in the figures are expressed as mean±s.e.mean. ED₅₀ values were determined using a non-linear regression analysis (Prism 2, GraphPad Software Inc., San Diego, Calif., USA). Statistical significance (p<0.05) was determined using a one-way analysis of variance (ANOVA) followed by a Student Newman-Keuls post hoc test for multiple comparisons between groups.

For the cultured DRG studies, the mean±s.e.mean of CGRP-immunopositive cells was obtained from 4 wells per treatment group in each of 3 separate cell cultures. Data are expressed as a percent of saline control wells. Statistical significance between treatment groups and controls was determined using a one-way analysis of variance followed by a Student Newman-Keuls post hoc test for multiple comparisons between groups (Prism 2, GraphPad Software Inc.). P<0.05 was considered significant.

What is claimed is:

1. A composition comprising an opioid receptor agonist in an amount effective to produce a therapeutic effect and a cannabinoid receptor antagonist in an amount effective to potentiate a therapeutic activity of an opioid receptor agonist and/or inhibit, delay, reduce and/or reverse tolerance to the opioid receptor agonist.

2. The composition of claim 1 wherein the opioid receptor agonist is an opioid.

3. The composition of claim 1 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2,D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, tramadol, dezocine, etorphine, tilidine, loperamide, diphenoxylate, paregoric and nalorphine.

4. The composition of claim 1 wherein the cannabinoid receptor antagonist is selected from the group consisting of SR 141716, AM-251, LY320135, and SR 144528.

5. The composition of claim 1 wherein the opioid receptor agonist is morphine and the cannabinoid receptor antagonist is AM-251.

6. The composition of claim 1 wherein the opioid receptor agonist is morphine and the cannabinoid receptor antagonist is SR 141716.

7. A method for potentiating a therapeutic activity of an opioid receptor agonist in a subject, the method comprising administering an opioid receptor agonist to the subject and administering a cannabinoid receptor antagonist to the subject in an amount effective to potentiate the therapeutic activity of the opioid receptor agonist and/or inhibit, delay, reduce and/or reverse tolerance to the opioid receptor agonist.

8. The method of claim 7 wherein the opioid receptor agonist is an opioid.

9. The method of claim 7 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2,D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, tramadol, dezocine, etorphine, tilidine, loperamide, diphenoxylate, paregoric and nalorphine.

10. The method of claim 7 wherein the cannabinoid receptor antagonist is selected from the group consisting of SR 141716, AM-251, LY320135, and SR 144528.

11. A method for inhibiting, delaying or reducing development of acute tolerance to a therapeutic effect of an opioid receptor agonist in a subject, the method comprising administering the opioid receptor agonist to the subject and administering a cannabinoid receptor antagonist to the subject.

12. The method of claim 11 wherein the opioid receptor agonist is an opioid.

13. The method of claim 11 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2,D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, tramadol, dezocine, etorphine, tilidine, loperamide, diphenoxylate, paregoric and nalorphine.

14. The method of claim 11 wherein the cannabinoid receptor antagonist is selected from the group consisting of SR 141716, AM-251, LY320135, and SR 144528.

15. A method for inhibiting, delaying or reducing development of chronic tolerance to a therapeutic effect of an opioid receptor agonist in a subject, the method comprising administering the opioid receptor agonist to the subject and administering a cannabinoid receptor antagonist to the subject.

16. The method of claim 15 wherein the opioid receptor agonist is an opioid.

17. The method of claim 15 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2,D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, tramadol, dezocine, etorphine, tilidine, loperamide, diphenoxylate, paregoric and nalorphine.

18. The method of claim 15 wherein the cannabinoid receptor antagonist is selected from the group consisting of SR 141716, AM-251, LY320135, and SR 144528.

19. A method for reversing tolerance to a therapeutic effect of an opioid receptor agonist or restoring a therapeutic effect of an opioid receptor agonist in a subject, the method comprising administering to the subject receiving opioid receptor agonist therapy a cannabinoid receptor antagonist.

20. The method of claim 19 wherein the cannabinoid receptor antagonist is selected from the group consisting of SR 141716, AM-251, LY320135, and SR 144528.

21. A method for treating a subject suffering from a condition treatable with an opioid receptor agonist, the method comprising administering an opioid receptor agonist to the subject in an amount effective to produce a therapeutic effect and administering a cannabinoid receptor antagonist to the subject in an amount effective to potentiate a therapeutic activity of the opioid receptor agonist and/or inhibit, delay, reduce and/or reverse tolerance to the opioid receptor agonist.

22. The method of claim 21 wherein the opioid receptor agonist is an opioid.

23. The method of claim 21 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2,D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, tramadol dezocine, etorphine, tilidine, loperamide, diphenoxylate, paregoric and nalorphine.

24. The method of claim 21 wherein the cannabinoid receptor antagonist is selected from the group consisting of SR 141716, AM-251, LY320135, and SR 144528.

25. The method of claim 21 wherein the subject is suffering from pain, coughing, diarrhea, pulmonary edema or addiction to an opioid receptor agonist.

26. The method of claim 25 wherein the pain is acute or chronic post-surgical pain, obstetrical pain, acute inflammatory pain, chronic inflammatory pain, pain associated with multiple sclerosis or cancer, pain associated with trauma, pain associated with migraines, neuropathic pain, central pain or a chronic pain syndrome of a non-malignant origin, or chronic back pain.

27. The method of claim 21 wherein the subject is treated for a condition treatable with an opioid receptor agonist without substantial undesirable side effects.

28. A method for treating a subject suffering from a condition treatable with an opioid receptor agonist comprising administering to a subject receiving opioid receptor agonist therapy a cannabinoid receptor antagonist in an amount effective to potentiate a therapeutic activity of the opioid receptor agonist and/or inhibit, delay, reduce and/or reverse tolerance to the opioid receptor agonist.

29. The method of claim 28 wherein the cannabinoid receptor antagonist is selected from the group consisting of SR 141716, AM-251, LY320135, and SR 144528.

30. The method of claim 28 wherein the subject is suffering from pain, coughing, diarrhea, pulmonary edema or addiction to an opioid receptor agonist.

31. The method of claim 30 wherein the subject is suffering from acute or chronic post-surgical pain, obstetrical pain, acute inflammatory pain, chronic inflammatory pain, pain associated with multiple sclerosis or cancer, pain associated with trauma, pain associated with migraines, neuropathic pain, central pain or chronic pain syndrome of a non-malignant origin.
